(19) United States
(12) Patent Application Publication
(10) Pub. No.: US 2022/0338840 A1
(43) Pub. Date: Oct. 27, 2022

(54) ULTRASOUND DIAGNOSTIC SYSTEM AND ULTRASOUND DIAGNOSTIC APPARATUS

(71) Applicant: FUJIFILM Healthcare Corporation, Kashiwa-shi (JP)

(72) Inventors: Akira Kusakabe, Chiba (JP); Koji Waki, Chiba (JP); Eiji Kasahara, Chiba (JP); Suguru Ishiguro, Chiba (JP)

(21) Appl. No.: 17/720,564

(22) Filed: Apr. 14, 2022

(30) Foreign Application Priority Data
Apr. 23, 2021 (JP) 2021-073325

(51) Int. Cl. A61B 8/00; A61B 8/08

(52) U.S. Cl. CPC A61B 8/463; A61B 8/465; A61B 8/5207; A61B 8/565; A61B 8/54

(57) ABSTRACT

When a basic mode is selected, a basic processing block in an ultrasound diagnostic apparatus operates in real time, to generate a first display image array, and the first display image array is displayed on a display. When an extended mode is selected, necessary information is transferred from the ultrasound diagnostic apparatus to an external information processing apparatus, and an extended processing block in the external information processing apparatus operates in real time, to generate a second display image array. The second display image array is displayed on the display.

### CROSS REFERENCE TO RELATED APPLICATION

This application claims priority to Japanese Patent Application No. 2021-073325 filed on Apr. 23, 2021, which is incorporated herein by reference in its entirety including the specification, claims, drawings, and abstract.

### TECHNICAL FIELD

The present disclosure relates to an ultrasound diagnostic system, and in particular, to an ultrasound diagnostic system formed from an ultrasound diagnostic apparatus and an external information processing apparatus.

### BACKGROUND

In an ultrasound diagnostic apparatus, a processor is responsible for executing a large number of functions related to processing, calculation, and control. Processing capability of the processor is finite, and, when a new function is added to the ultrasound diagnostic apparatus, the processing capability of the processor becomes an issue. For example, during ultrasound diagnosis, if one wishes to use a three-dimensional image formation function, a machine learning-type image analysis function, a diagnosis assisting function, or the like, the above-described problem tends to occur. Developers and users have needs for equipping the ultrasound diagnosis apparatus with extended functions other than the basic functions, but the finiteness of the processing capability of the processor is becoming an obstacle against satisfying these needs.

A configuration may be employed in which an external information processing apparatus is additionally connected to the ultrasound diagnostic apparatus, to form an ultrasound diagnostic system, so that the processing capability of the external information processing apparatus can be utilized. That is, it becomes possible to cause the external information processing apparatus to carry the extended function. For this purpose, the ultrasound diagnostic apparatus and the external information processing apparatus must cooperate properly.

When the identical peripheral devices (such as display devices) are provided individually for the ultrasound diagnostic apparatus and for the external information processing apparatus, a problem may be caused in that an amount of resources is increased (along with cost). There also is a problem in that, at the periphery of the ultrasound diagnostic apparatus, there is insufficient spacing for placing the external information processing apparatus with the peripheral device.

JP 2004-8535 A (“Document 1”) discloses an ultrasound diagnostic system with an external information processing apparatus. The external information processing apparatus is a desktop-type computer, and comprises a computer body and a display. In the external information processing apparatus, measurement is performed with respect to image data transferred from an ultrasound diagnostic apparatus, and a result of the measurement is then transferred to the ultrasound diagnostic apparatus. Document 1 does not describe real-time external processing performed at the external information processing apparatus, or real-time display of a result of the external processing in the ultrasound diagnosis apparatus.

An advantage of the present disclosure lies in reducing the amount of resources when constructing the ultrasound diagnostic system. Alternatively, an advantage of the present disclosure lies in realization of a novel ultrasound diagnostic system having a basic processing block and an extended processing block which function in real time.

### SUMMARY

According to one aspect of the present disclosure, there is provided an ultrasound diagnostic system comprising: an ultrasound diagnostic apparatus; and an external information processing apparatus that is connected to the ultrasound diagnostic apparatus, and that executes an extended function which is beyond a basic function provided in the ultrasound diagnostic apparatus. The ultrasound diagnostic apparatus comprises a first image generator which generates a first image as a real-time image including an ultrasound image, a display controller which selects the first image when a basic mode is selected, and selects a second image generated by the external information processing apparatus when an extended mode is selected, and a display which displays the image selected by the display controller. The external information processing apparatus includes a second image generator which generates the second image as a real-time image based on the ultrasound image.

According to another aspect of the present disclosure, there is provided an ultrasound diagnostic apparatus to which an external information processing apparatus which realizes an extended function is connected. The extended function is a function which is beyond a basic function provided in the ultrasound diagnostic apparatus. The ultrasound diagnostic apparatus comprises: a unit which selects a basic mode and an extended mode; a unit which generates a first image as a real-time image including an ultrasound image; a unit which selects the first image when the basic mode is selected, and selects a second image generated by the external information processing apparatus when the extended mode is selected; and a unit which displays the image which is selected. The second image is a real-time image generated by the extended function based on the ultrasound image.

### BRIEF DESCRIPTION OF DRAWINGS

Embodiment(s) of the present disclosure will be described based on the following figures, wherein:

FIG. 1 is a block diagram showing an ultrasound diagnostic apparatus according to an embodiment of the present disclosure;

FIG. 2 is a diagram showing an external information processing apparatus (external apparatus) equipped on a body;

FIG. 3 is a diagram showing an example of an operation panel;

FIG. 4 is a diagram showing an example of an ultrasound probe;

FIG. 5 is a diagram showing a first display image and a second display image;

FIG. 6 is a flowchart showing an example operation of the ultrasound diagnostic apparatus;

FIG. 7 is a flowchart showing an operation when the power is turned ON;

FIG. 8 is a flowchart showing an operation during shut-down; and

FIG. 9 is a diagram showing an ultrasound diagnostic system according to an alternative configuration.

### DESCRIPTION OF EMBODIMENTS

An embodiment of the present disclosure will now be described with reference to the drawings.

### (1) Overview of Embodiment

An ultrasound diagnostic system according to an embodiment of the present disclosure comprises an ultrasound diagnostic apparatus and an external information processing apparatus. The external information processing apparatus is an apparatus which is connected to the ultrasound diagnostic apparatus, and executes an extended function which is beyond a basic function provided in the ultrasound diagnostic apparatus. The ultrasound diagnostic apparatus comprises a first image generator, a display controller, and a display. The first image generator generates a first image as a real-time image including an ultrasound image. The display controller selects the first image when a basic mode is selected, and selects a second image generated by the external information processing apparatus when an extended mode is selected. The display displays the image selected by the display controller. The external information processing apparatus includes a second image generator which generates the second image as a real-time image based on the ultrasound image.

According to the structure described above, there can be constructed an ultrasound diagnostic system which has a basic processing block and an extended processing block which function in real time. The basic processing block is a processing block originally provided in the ultrasound diagnostic apparatus, and includes the first image generator. The extended processing block is a processing block realized by the addition of the external information processing apparatus, and includes the second image generator. When the basic mode is selected, the basic processing block functions, and the first image is generated and displayed in real time. On the other hand, when the extended mode is selected, the extended processing block functions, and the second image is generated and displayed in real time. The first image and the second image are selectively displayed on the display provided in the ultrasound diagnostic apparatus.

In the above-described structure, the real-time image is a video image generated simultaneously with transmission and reception of ultrasound, and is an image reflecting reception information (echo information) acquired at each point in time. By removing the display from the external information processing apparatus, it is possible to reduce the amount of resources corresponding to the display, and to consequently reduce cost. Even when the external information processing apparatus has a display, displaying the second image on the display of the ultrasound diagnostic apparatus enables easier interpretation of the image.

As the name indicates, the external information processing apparatus is an apparatus which can execute an information processing as a single entity. In an embodiment of the present disclosure, the external information processing apparatus comprise a power supply, a processor, an operating system (OS), or the like, similar to the ultrasound diagnostic apparatus. The extended function is a function other than the basic function. Even when the extended function corresponds to a heavy-load process, because the processing is carried by the processor in the external processing apparatus, an excessive load on the processor in the ultrasound diagnostic apparatus can be avoided. Alternatively, the basic function provided in the ultrasound diagnostic apparatus may be equipped also in the external information processing apparatus in an overlapping manner.

In an embodiment of the present disclosure, the external information processing apparatus is placed or connected with respect to the ultrasound diagnostic apparatus. For example, the external information processing apparatus may be placed on a base provided on the body of the ultrasound diagnostic apparatus, or the external information processing apparatus may be connected to a side surface of the body of the ultrasound diagnostic apparatus. Presuming such a placement form, the shape of the external information processing apparatus may be a box shape of a relatively small size. By removing the display and the inputting unit from the external information processing apparatus, the size of the overall external information processing apparatus may be reduced, and the overall external information processing apparatus may be simplified.

In an embodiment of the present disclosure, the ultrasound diagnostic apparatus has a mode selection unit for a user to select the basic mode and the extended mode. The mode selection unit may be formed from a physical operator, a virtual operator (for example, an icon), or the like. Alternatively, an operation mode may be automatically selected.

In an embodiment of the present disclosure, the ultrasound diagnostic apparatus comprises an inputting unit and a transfer control unit. In the extended mode, the transfer control unit transfers to the external information processing apparatus particular input information which is input via the inputting unit and which is necessary for execution of the extended function. According to this configuration, it becomes unnecessary to always provide the inputting unit on an external inputting device.

In an embodiment of the present disclosure, the ultrasound diagnostic apparatus comprises a transfer control unit. In the extended mode, the transfer control unit transfers to the external information processing apparatus particular parameter information which is held in the ultrasound diagnostic apparatus and which is necessary for execution of the extended function. According to this configuration, the extended function provided in the external information processing apparatus can be appropriately operated in the external information processing apparatus.

In an embodiment of the present disclosure, the ultrasound diagnostic apparatus comprises an associated device and a transfer control unit. The transfer control unit comprises a transfer control unit which, in the extended mode, transfers to the external information processing apparatus particular associated information which is generated by the associated device and which is necessary for execution of the extended function. According to this configuration, it becomes unnecessary to directly output the associated information from the associated device to the external information processing apparatus.

In an embodiment of the present disclosure, the ultrasound image is transferred from the ultrasound diagnostic apparatus to the external information processing apparatus via a first communication line. The second image is transferred from the external information processing apparatus to the ultrasound diagnostic apparatus via a second communication line different from the first communication line. With combined use of a plurality of communication lines, a large amount of data can be quickly exchanged. The first communication line may be formed by a general network, and the second communication line may be formed by a general video signal line.

In an embodiment of the present disclosure, the ultrasound diagnostic apparatus comprises a switch, and a power supply control unit. The switch is a switch for a power-ON operation and for a power-OFF operation. The power supply control unit controls starting-up of the ultrasound diagnostic apparatus and the external information processing apparatus when the power-ON operation is executed. In addition, the power supply control unit controls shutting-down of the ultrasound diagnostic apparatus and the external information processing apparatus when the power-OFF operation is executed. According to this configuration, the starting-up and the shutting-down of the two apparatuses can be appropriately linked together.

An ultrasound diagnostic apparatus according to an embodiment of the present disclosure is an ultrasound diagnostic apparatus to which an external information processing apparatus which realizes an extended function is connected. The extended function is a function which is beyond a basic function provided in the ultrasound diagnostic apparatus. The ultrasound diagnostic apparatus comprises a unit which selects a basic mode and an extended mode, a unit which generates a first image as a real-time image including an ultrasound image, a unit which selects the first image when the basic mode is selected and which selects a second image generated by the external information professing apparatus when the extended mode is selected, and a unit which displays the image which is selected. The second image is a real-time image generated by the extended function based on the ultrasound image.

### (2) Details of Embodiment

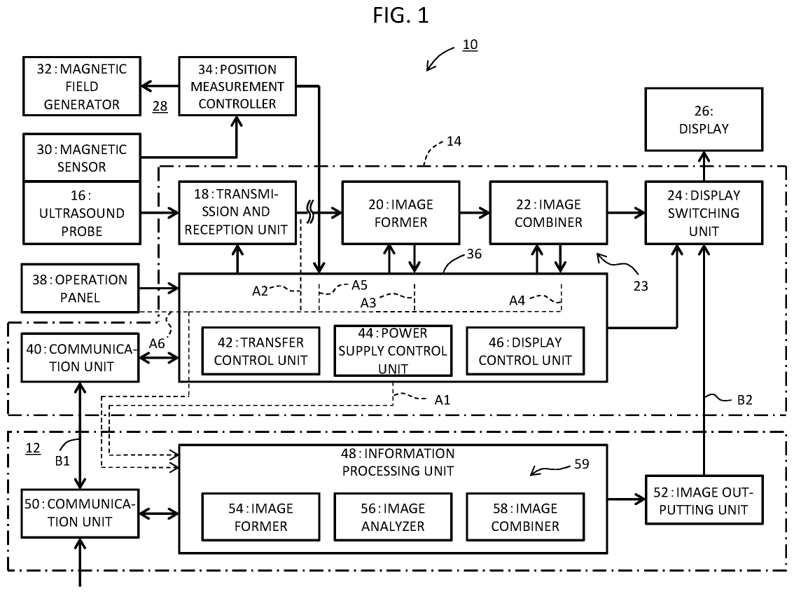

FIG. 1 shows an ultrasound diagnostic system according to an embodiment of the present disclosure. The ultrasound diagnostic system is, for example, a medical system which is mounted in a medical facility, and which generates and displays an ultrasound image in real time based on information acquired by transmission of ultrasound to a living body and reception of a reflected wave from the inside of the living body.

The ultrasound diagnostic system is formed from an ultrasound diagnostic apparatus 10 and an external information processing apparatus (hereinafter referred to as “external apparatus”) 12. The ultrasound diagnostic apparatus 10 is one type of an information processing apparatus. Thus, from this viewpoint, the ultrasound diagnostic system may be described as a system having two information processing apparatuses. The external apparatus 12 has an extended function which is beyond a basic function provided in the ultrasound diagnostic apparatus 10.

The ultrasound diagnostic system has a basic mode and an extended mode. A user (such as a doctor, an examining technician, or the like) can select either of the modes. When the basic mode is selected, a basic processing block in the ultrasound diagnostic apparatus 10 operates in real time. When the extended mode is selected, an extended processing block in the external apparatus 12 operates in real time. That is, one of the processing blocks operates simultaneously with the transmission and reception of the ultrasound. Alternatively, the two processing blocks may operate in parallel to each other.

The ultrasound diagnostic apparatus 10 comprises a body 14. An ultrasound probe 16 is detachably connected to the body 14. As the ultrasound probe 16, there are known a probe which is used in contact with a body surface of an examination target, a probe which is used by being inserted into a body cavity of the examination target, and the like. The ultrasound probe 16 comprises, for example, a transducer array including a plurality of transducers which are one-dimensionally arranged. An ultrasound beam is formed by the transducer array, and is electronically scanned. Alternatively, a transducer array including a plurality of transducers which are two-dimensionally arranged may be provided, or a plurality of transducer arrays orthogonal to each other may be provided in the ultrasound probe 16.

A transmission and reception unit 18 supplies a plurality of transmission signals to the transducer array in parallel to each other, during transmission. During reception, the transmission and reception unit 18 applies a predetermined processing (such as amplification, A/D conversion, delay, addition, or the like) to a plurality of reception signals which are output from the transducer array in parallel to each other. With this process, beam data are generated. With repetition of electronic scanning of the ultrasound beam, a reception frame data array is output from the transmission and reception unit 18.

The reception frame data array is formed from a plurality of sets of reception frame data which are arranged on a time axis. Each individual set of reception frame data is formed from a plurality of sets of beam data arranged in the direction of the electronic scan. Each individual sets of beam data is formed from a plurality of sets of echo data arranged in the depth direction. A beam data processor is provided downstream of the transmission and reception unit 18, but illustration of the beam data processor is omitted.

An image former 20 generates a display frame data array based on the reception frame data array, and is more specifically formed from a DSC (Digital Scan Converter). The DSC is formed from a processor having a coordinate conversion function, a pixel interpolation function, or the like. Each individual set of display frame data corresponds to a tomographic image.

An image combiner 22 combines a graphic image or the like to the tomographic image, to thereby generate a display image (a first display image or a first image). The image combiner 22 outputs a first display frame data array as a display frame data array after the combining.

A display switching unit 24 selects one display frame data array (display image array) among the first display frame data array (first display image array) and a second display frame data array (second display image array) which is output from the external apparatus 12, and outputs the selected display frame data array to a display 26, under control of a display control unit 46 to be described later.

On the display 26, the selected display image array is displayed as a video image in real time. Alternatively, a display image of a particular time phase may be displayed as a still image in a frozen state in which transmission and reception is suspended. The display 26 is formed from a liquid crystal display, an organic EL device, or the like. The image former 20 and the image combiner 22 correspond to the basic processing block, and form a first image generator 23.

The illustrated ultrasound diagnostic apparatus 10 has a position measurement unit 28 serving as an associated device. The position measurement unit 28 is formed from a magnetic sensor 30, a magnetic field generator 32, and a position measurement controller 34. The magnetic sensor 30 is attached on the ultrasound probe 16. The magnetic field generator 32 generates a magnetic field for identifying three-dimensional coordinate information, and the magnetic field is detected by the magnetic sensor 30. The position measurement controller 34 controls operations of the magnetic field generator 32. The position measurement controller 34 also calculates position information for identifying a position and an orientation of the magnetic sensor 30 in the three-dimensional space, based on an output signal of the magnetic sensor. The position information is output from the position measurement controller to an information processing unit 36. Examples of other associated devices include an electrocardiograph, a puncture guiding mechanism, and the like.

An operation panel 38 includes a plurality of switches, a plurality of knobs, a trackball, a touch screen panel, or the like. The operation panel 38 functions as an inputting unit. A communication unit 40 is connected to the information processing unit 36.

The information processing unit 36 executes control of operations of various elements of the ultrasound diagnostic apparatus. In addition, the information processing unit 36 executes control necessary for causing the external apparatus 12 to function and cooperate. The information processing unit 36 is formed from a processor which executes a program. The processor is more specifically a CPU (Central Processing Unit). In FIG. 1, a plurality of functions realized by the processor are represented by a plurality of blocks. That is, the information processing unit 36 comprises a transfer control unit 42, a power supply control unit 44, and the display control unit 46.

The transfer control unit 42 executes control to transfer information from the ultrasound diagnostic apparatus 10 to the external apparatus 12 when the extended mode is selected. The information which is transferred is information necessary for execution of the extended function, and is, for example, parameter information, a frame data array, input information, associated information, or the like.

More specifically, of all parameter information which is set in the ultrasound diagnostic apparatus 10, particular parameter information necessary for execution of the extended function is transferred to the external apparatus 12 (refer to A1). Further, of all of the input information which is input to the ultrasound diagnostic apparatus 10, particular input information necessary for execution of the extended function is transferred to the external apparatus 12 (refer to A6). Moreover, of the associated information which is input to the ultrasound diagnostic apparatus 10, particular associated information necessary for execution of the extended function is transferred to the external apparatus 12 (refer to A5). In the present embodiment, the associated information is the position information.

The frame data array which is transferred is typically the display frame data array which is output from the image former 20; that is, a tomographic image array serving as an ultrasound image array (refer to A3). The tomographic image array is output from the ultrasound diagnostic apparatus 10 to the external apparatus 12 in real time. Alternatively, in place of such a display frame data array, a reception frame data array which is output from the transmission and reception unit 18 or the combined display frame data array which is output from the image combiner 22 may be transferred (refer to A2 and A4).

The transfer control unit 42 has management data for identifying the information necessary for execution of the extended function in the external apparatus 12. When the extended function is changed, added, deleted, or the like, the management data are correspondingly changed, added, deleted, or the like.

Alternatively, the transfer control unit 42 may attach a timestamp (time information) on each frame data. Alternatively, a management unit may be provided for synchronizing the ultrasound diagnostic apparatus 10 and the external apparatus 12.

The power supply control unit 44 controls starting-up of the power supply of the ultrasound diagnostic apparatus 10 and the external apparatus 12 when a power button in the ultrasound diagnostic apparatus 10 is operated to be turned ON, and controls shutting-down of the power supply of the ultrasound diagnostic apparatus 10 and the external apparatus 12 when the power button is operated to be turned OFF.

The display control unit 46 selects a display image to be displayed on the display 26, through the display switching unit 24. The display control unit 46 selects the first display frame data array (first display image array) which is output from the image combiner 22 when the basic mode is selected, and selects the second display frame data array (second display image array) which is sent from the external apparatus 12 when the extended mode is selected.

The ultrasound diagnostic apparatus 10 includes a power supply (not shown), the above-described processor (CPU), and an operating system (OS) executed by the processor.

The external apparatus 12 will next be described. The external apparatus 12 comprises an information processing unit 48, a communication unit 50, and an image outputting unit 52. The information processing unit 48 executes a plurality of functions including the extended function. These functions are represented by a plurality of blocks in FIG. 1.

Specifically, in the illustrated example structure, the information processing unit 48 comprises an image former 54, an image analyzer 56, and an image combiner 58. Similar to the ultrasound diagnostic apparatus 10, the external apparatus 12 is an information processing apparatus having a power supply, a processor (CPU), and an operating system. More specifically, the external apparatus 12 is a computer.

In the illustrated example structure, the ultrasound diagnostic apparatus 10 and the external apparatus 12 are connected to each other via a first communication line B1 and a second communication line B2. Specifically, the communication unit 40 and the communication unit 50 are connected to each other via the first communication line B1, and the image outputting unit 52 and the display switching unit 24 are connected to each other via the second communication line B2. The first communication line B1 is, for example, a wired or wireless network according to a general standard. The second communication line B2 is formed from, for example, a general video signal line.

The image former 54 is, for example, a module which forms a three-dimensional image. For example, the image former 54 forms a three-dimensional image three-dimensionally representing a tissue in the living body in real time, based on the display frame data array and the position data array generated during the movement of the probe. The three-dimensional image is physically a two-dimensional image.

The image analyzer 56 is a module, for example, which applies image analysis for assisting diagnosis on the display frame data array. Alternatively, a machine learning-type estimator may be provided as the image analyzer 56. For example, the image analyzer 56 identifies a lesion site, and generates an image having a marker showing the lesion site. Alternatively, the image analyzer 56 may be formed from a CNN (Convolutional Neural Network).

Various software modules satisfying various needs of the designer and the user may be incorporated in the information processing unit 48. During the operation of the external apparatus 12, a certain load is caused in the processor in the ultrasound diagnostic apparatus 10 for controlling transfer, but the increase in the load is relatively very small.

The image combiner 58 is a module which combines a plurality of images generated or formed in the information processing unit 48, to generate the second display image. In reality, the image combiner 58 generates the second display frame data array (second display image array) which changes dynamically, and the second display frame data array is output via the image outputting unit 52 to the ultrasound diagnostic apparatus 10.

The external apparatus 12 has a function to receive data acquired by other medical apparatuses. Examples of the other medical apparatuses include an MRI apparatus, an X-ray CT apparatus, other ultrasound diagnostic apparatuses, and the like. The image former 54, the image analyzer 56, and the image combining unit 58 form the extended processing block. The image former 54, the image analyzer 56, and the image combining unit 58 correspond to a second image generator 59. An alternatively configuration may be employed in which the first display image array and the second display image array are combined by the display switching unit 24. In this case, the timestamp or the like may be utilized for synchronizing the two display image arrays.

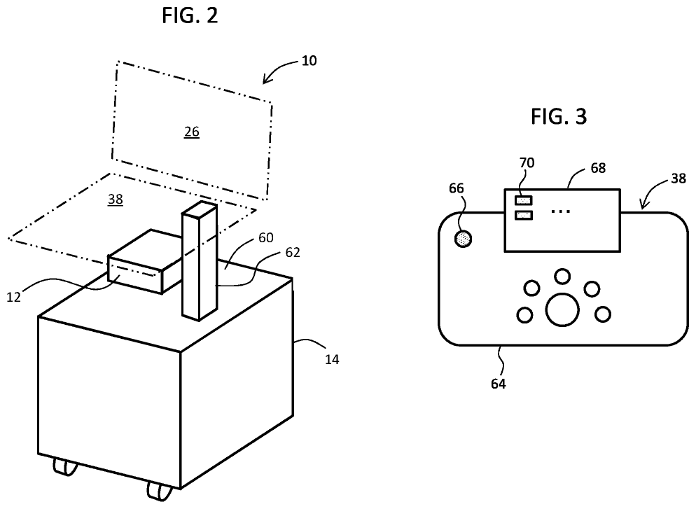

FIG. 2 schematically shows the ultrasound diagnostic system. The ultrasound diagnostic apparatus 10 has the body 14. The body 14 has a plurality of casters, and houses the power supply and a plurality of the electronic boards in the body 14. A support column 62 supports the operation panel 38 and the display 26. An upper surface of the body 14 includes a base surface, and the external apparatus 12 is detachably mounted on the base surface. In the illustrated example structure, the external apparatus 12 has a box-shape form. Alternatively, the external apparatus 12 may be mounted on a side surface of the body 14 or on a lower surface of the operation panel 38.

The ultrasound diagnostic apparatus 10 and the external apparatus 12 are connected to each other via a plurality of cables. The plurality of cables may include a communication cable, a video signal cable, a power supply cable, or the like. Electric power is supplied from the ultrasound diagnostic apparatus 10 to the external apparatus 12. Alternatively, wireless communication may be executed between the ultrasound diagnostic apparatus 10 and the external apparatus 12. In this case, a wireless base unit may be provided on the ultrasound diagnostic apparatus 10.

FIG. 3 schematically shows the operation panel 38. On the operation panel 38, a plurality of buttons, a plurality of knobs, a trackball, or the like are provided. Further, a power button (power switch) 66 and a touch screen panel 68 are provided.

The touch screen panel 68 is a layered structure of a multi-touch panel and a display panel. A plurality of virtual buttons are displayed on the touch screen panel 68, including a mode switching button 70. With an operation of the mode switching button 70, one of the basic mode and the extended mode can be selected.

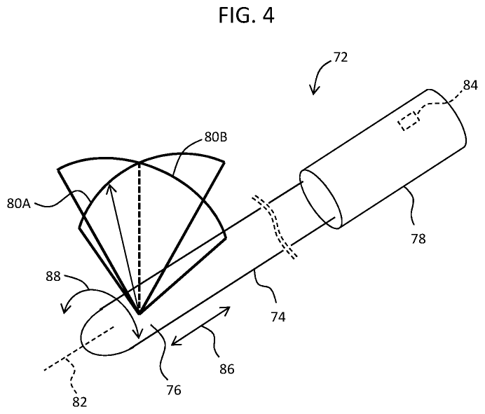

FIG. 4 shows an example of an ultrasound probe. An ultrasound probe 72 which is illustrated is a transrectal probe. The ultrasound probe 72 includes an insertion unit 74 inserted into a rectum, and an operation unit 78. Two transducer arrays which are orthogonal to each other are provided in the insertion unit 74, and one of the transducer arrays can be selected for use. With the two transducer arrays, a first scanning plane 80A parallel to a center axis 82 and a second scanning plane 80B orthogonal to the center axis 82 are selectively formed. The first scanning plane 80A and the second scanning plane 80B translate along the center axis 82 by a front-and-rear motion 86 of the ultrasound probe 72, and rotate about the center axis 82 by a rotational motion 88 of the ultrasound probe 72.

In the illustrated example structure, a magnetic sensor 84 is provided in the operation unit 78. Positions and orientations of the first scanning plane 80A and the second scanning plane 80B in the three-dimensional space are identified based on an output signal of the magnetic sensor 84. With an operation of a predetermined button provided on the operation panel, a scanning plane to actually function is selected from the first scanning plane 80A and the second scanning plane 80B. When the extended mode is selected, information of the selected scanning plane and depth information of the scanning plane are transferred from the ultrasound diagnostic apparatus to the external apparatus.

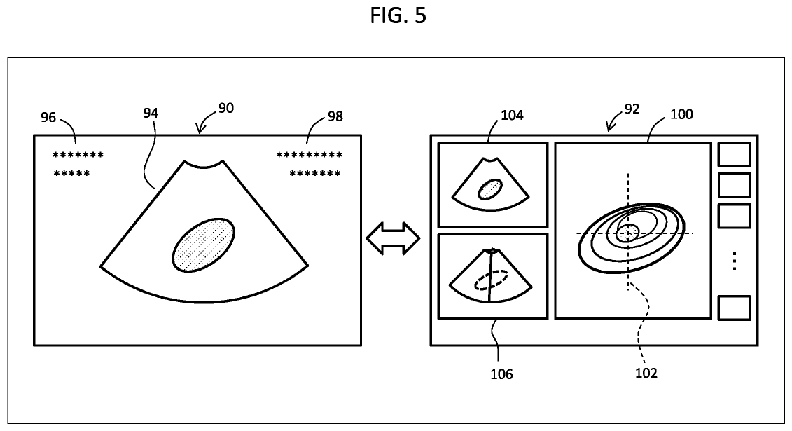

FIG. 5 shows an example display. In a state in which the basic mode is selected, the first display image array is displayed on the display. FIG. 5 shows a particular first display image 90 in the first display image array. In a state in which the extended mode is selected, the second display image array is displayed on the display. FIG. 5 shows a particular second display image 92 in the second display image array.

The first display image 90 includes a tomographic image 94 serving as an ultrasound image, and also includes a graphic image including text information 96 and 98. In the illustrated example structure, the second display image 92 includes a three-dimensional image 100, a tomographic image 104, an MRI image 106, or the like. The three-dimensional image 100 is a real-time image constructed based on the display frame data array and the position data array, and includes a graphic image for assisting examination, diagnosis, and interpretation. FIG. 5 shows a marker 102 as an element forming the graphic image.

The tomographic image 104 is an ultrasound image serving as a real-time image. The MRI image is a still image corresponding to the three-dimensional image. The MRI image corresponding to the three-dimensional image is generated based on MRI volume data. In this process, position information is used.

The first display image 90 and the second display image 92 which are illustrated are merely exemplary. With an active use of the external apparatus, a highly advanced image processing result may be acquired during the ultrasound examination without exchanging the ultrasound diagnostic apparatus, and a highly advanced diagnosis assistance may be provided.

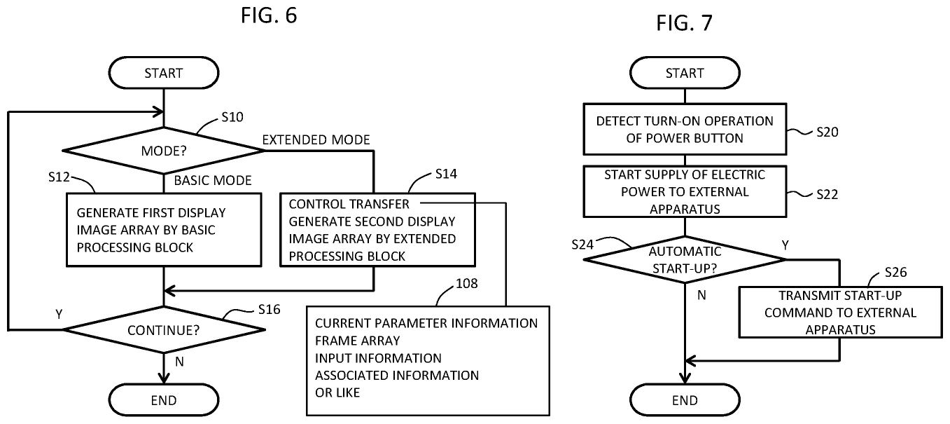

FIG. 6 shows an example operation of the ultrasound diagnostic apparatus. In S10, the mode which is being selected is determined. When the selected mode is determined as the basic mode, S12 is executed. That is, the basic processing block functions, and the first display image array is generated and displayed in real time. On the other hand, when the selected mode is determined as the extended mode in S10, S14 is executed. In S14, information necessary for execution of the extended function is transferred from the ultrasound diagnostic apparatus to the external apparatus, the extended processing block functions, and the second display image array is generated. The second display image array is transferred from the external apparatus to the ultrasound diagnostic apparatus, and is displayed on the display in real time. Examples of the information transferred from the ultrasound diagnostic apparatus to the external apparatus include the parameter information, the frame array, the input information, the associated information, and the like, as shown by reference numeral 108. In S16, it is determined whether or not to continue this process.

FIG. 7 shows control executed by the power supply control unit upon the power-ON operation. In S20, the turn-ON operation of the power button is detected. With this detection, supply of electric power to various units in the ultrasound diagnostic apparatus is started. In S22, supply of electric power to the external apparatus is started. In S24, it is judged whether or not an automatic start-up mode is set for the external apparatus. When the automatic start-up mode is set, in S26, a start-up command is transmitted to the external apparatus. With this process, the operating system or the like provided in the external apparatus is started up.

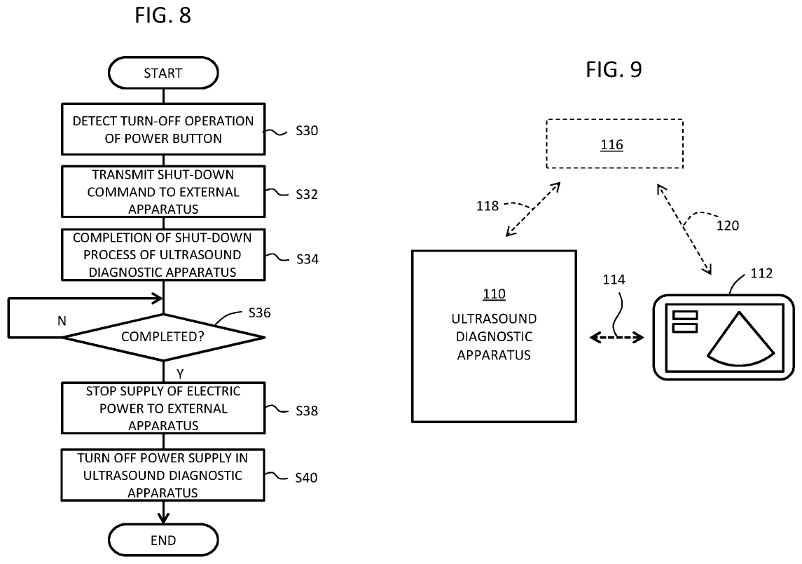

FIG. 8 shows control executed by the power supply control unit during turning-OFF of the power supply. When a turn-OFF operation of the power button is detected in S30, a shutting-down process of the ultrasound diagnostic apparatus is started, and, in S32, a shut-down command is transmitted to the external apparatus. In S34, completion of the shut-down process at the ultrasound diagnostic apparatus is determined. In S36, completion of the shut-down process at the external apparatus is determined. After the completion of these processes is confirmed, the supply of the electric power to the external apparatus is stopped in S38, and the power supply in the ultrasound diagnostic apparatus is turned OFF in S40. According to the above-described control, it is possible to prevent occurrence of a situation in which the power supply of the ultrasound diagnostic apparatus is turned OFF during the shut-down process of the external apparatus.

FIG. 9 shows an alternative configuration. An ultrasound diagnostic apparatus 110 is connected to an external apparatus 112 via a wired or wireless communication line 114. The external apparatus 112 is a tablet terminal having a touch screen panel. That is, the external apparatus 112 is a terminal having an inputting unit and a display. When the extended mode is selected, necessary information is transferred from the ultrasound diagnostic apparatus to the external apparatus 112, and an image generated by the external apparatus 112 is displayed on a display of the ultrasound diagnostic apparatus 110. For example, the ultrasound diagnostic apparatus 110 and the external apparatus 112 may be connected to each other via a wireless relay 116 (refer to reference numerals 118 and 120).

In the structure shown in FIG. 1, when the extended mode is selected, the operation of the basic processing block may be stopped or continued. Even when the basic processing block operates, the display image to be displayed is the second display image generated by the external apparatus. Alternatively, the first display image and the second display image may be displayed in parallel to each other, or an image in which the first and second display images are combined may be displayed.

1. An ultrasound diagnostic system comprising:
an ultrasound diagnostic apparatus; and
an external information processing apparatus that is connected to the ultrasound diagnostic apparatus, and that executes an extended function which is beyond a basic function provided in the ultrasound diagnostic apparatus, wherein
the ultrasound diagnostic apparatus comprises:
a first image generator which generates a first image as a real-time image including an ultrasound image;
a display controller which selects the first image when a basic mode is selected, and selects a second image generated by the external information processing apparatus when an extended mode is selected; and
a display which displays the image selected by the display controller, and
the external information processing apparatus includes a second image generator which generates the second image as a real-time image based on the ultrasound image.

2. The ultrasound diagnostic system according to claim 1, wherein
the external information processing apparatus is placed or connected with respect to the ultrasound diagnostic apparatus.

3. The ultrasound diagnostic system according to claim 1, wherein
the ultrasound diagnostic apparatus comprises a mode selector for a user to select the basic mode and the extended mode.

4. The ultrasound diagnostic system according to claim 1, wherein
the ultrasound diagnostic apparatus comprises an inputting unit, and
the ultrasound diagnostic apparatus comprises a transfer control unit which, in the extended mode, transfers to the external information processing apparatus particular input information which is input via the inputting unit and which is necessary for execution of the extended function.

5. The ultrasound diagnostic system according to claim 1, wherein
the ultrasound diagnostic apparatus comprises a transfer control unit which, in the extended mode, transfers to the external information processing apparatus particular parameter information which is held in the ultrasound diagnostic apparatus and which is necessary for execution of the extended function.

6. The ultrasound diagnostic apparatus according to claim 1, wherein
the ultrasound diagnostic apparatus comprises an associated device, and
the ultrasound diagnostic apparatus comprises a transfer control unit which, in the extended mode, transfers to the external information processing apparatus particular associated information which is generated by the associated device and which is necessary for execution of the extended function.

7. The ultrasound diagnostic system according to claim 1, wherein
the ultrasound image is transferred from the ultrasound diagnostic apparatus to the external information processing apparatus via a first communication line, and
the second image is transferred from the external information processing apparatus to the ultrasound diagnostic apparatus via a second communication line different from the first communication line.

8. The ultrasound diagnostic system according to claim 1, wherein
the ultrasound diagnostic apparatus comprises:
a switch for a power-ON operation and for a power-OFF operation; and
a power supply control unit which controls starting-up of the ultrasound diagnostic apparatus and the external information processing apparatus when the power-ON operation is executed, and controls shutting-down of the ultrasound diagnostic apparatus and the external information processing apparatus when the power-OFF operation is executed.

9. An ultrasound diagnostic apparatus to which an external information processing apparatus which realizes an extended function is connected, wherein
the extended function is a function which is beyond a basic function provided in the ultrasound diagnostic apparatus,
the ultrasound diagnostic apparatus comprises:
a unit which selects a basic mode or an extended mode;
a unit which generates a first image as a real-time image including an ultrasound image;
a unit which selects the first image when the basic mode is selected, and which selects a second image generated by the external information processing apparatus when the extended mode is selected; and
a unit which displays the image which is selected, and
the second image is a real-time image generated by the extended function based on the ultrasound image.
